(19) United States
(12) Patent Application Publication
(10) Pub. No.: US 2010/0184129 A1
(43) Pub. Date: Jul. 22, 2010

(54) SILICON MOULDED PART COMPRISING LUMINOUS BODIES

(75) Inventors: Jochen Dauth, Burghausen (DE); Christoph Muller-Rees, Pullach (DE); Christian Ochs, Burghausen (DE)

Correspondence Address: RATNERPRESTIA, P.O. BOX 980, VALLEY FORGE, PA, 19482, US

(73) Assignee: WACKER CHEMIE AG, Munich (DE)

(21) Appl. No.: 12/602,429

(22) PCT Filed: May 30, 2008
(86) PCT No.: PCT/EP2008/056665

§ 371 Date: Jan. 18, 2010

(30) Foreign Application Priority Data
Jun. 1, 2007 (DE) 10 2007 025 749.1

(51) Int. Cl. C12P 1/00; B01J 19/08; H01L 33/00

(52) U.S. Cl. 435/41; 204/157.15; 257/88; 438/26; 257/E33.056

(57) ABSTRACT

The invention relates to moulded parts containing a plurality of luminous bodies incorporated into a silicon matrix, the silicon matrix consisting of an inner soft silicon matrix A which is surrounded by at least one hard silicon matrix B. The silicon matrix B is optionally coated with a top coat C.

### CROSS REFERENCE TO RELATED APPLICATIONS

This is a U.S. National Phase application of PCT application number PCT/EP2008/056665, filed May 30, 2008, which claims priority benefit of German application number DE 10 2007 025 749.1 (filed Jun. 1, 2007), the content of such applications being incorporated herein by reference.

### FIELD OF THE INVENTION

The invention relates to illuminant-silicone moldings which comprise a plurality of illuminants embedded in a silicone matrix.

### BACKGROUND OF THE INVENTION

Illuminants referred to as LED illuminants or else light-emitting diodes are light-emitting electronic components. LED technology is notable in that illuminants are available over a wide range of the electromagnetic spectrum, the individual LED illuminants emitting very specifically within a narrow spectral range. This makes LED illuminants components of interest for many applications, for example for initiating chemical reactions and biological processes. Further advantages consist in low heat release, relatively high light yield and relatively long lifetime.

For protection from environmental influences, it is known that individual light-emitting diodes can be encapsulated with silicones: U.S. Pat. No. 6,916,889 B2 discloses encapsulating LEDs using crosslinkable epoxy-functional silicones. WO 2006/055196 A1 claims the encapsulation of LED illuminants with photopolymerizable silicone. EP 1 684 363 A2 describes the encapsulation of LEDs with crosslinkable silicone, wherein a first step involves encapsulating the LED chip with a crosslinked silicone rubber, and then applying a second layer of crosslinked silicone resin. DE 3019239 A1 describes the enveloping of individual semiconductor components with two layers, one of which consists of a hard polymer. What is described specifically is an envelope with a first, soft layer of silicone or epoxy resin, and a second, hard layer of silicone, epoxy resin or thermoplastic. In US 2005/0130336 A1, individual LEDs are encapsulated with a hard, transparent lenticular material, and the cavity formed between capsule and LED is filled by means of a needle with soft material, for example liquid silicone. An analogous method is described in DE 19945675 A1, wherein the individual LED chip is encapsulated with an optical epoxy resin lens, and the cavity is filled with liquid or gelatin-like silicone. EP 1657758 A2 describes applying one or more LEDs to a carrier and equipping each individual LED with an inner, soft lens and an outer, hard lens. Carrier and soft and hard lenses may be formed from silicone.

A disadvantage of the encapsulation of individual LED chips is the high complexity in terms of apparatus and process technology that is involved. LED chips which are protected effectively from environmental influences are obtained, but, where a plurality of LED chips connected to electrical conductors and provided with contacts are used, conductors and contacts are still exposed unprotected to environmental influences.

DE 102005050947 A1 describes a luminous element in which a plurality of LED chips are connected to one another and embedded in a plastic or glass casing filled with an inert material such as silicone. In US 2003/0042844 A1, a plurality of LED chips are arranged on the base of a trough-like frame and the trough is filled with epoxy resin. DE 3827083 A1 discloses the production of areal radiators, wherein a plurality of LEDs are mounted on a transparent mounting board, and silicone resin is cast onto each LED by means of a mold to form a reflector body, and the reflector bodies are finally coated with reflective material. DE 20 2006 001 561 U1 discloses pipe-shaped luminous bands, in which a plurality of light-emitting diodes connected via a cable are embedded in a silicone pipe.

A disadvantage of the shaped bodies described is in particular the complex production thereof and the low flexibility with regard to the shaping of the shaped bodies, for example in the case of the trough-like shaped bodies or the glass vessels filled with LEDs. Another disadvantage is the insufficient protection of conductive connecting elements between the illuminants and of contacts.

It was an object of the present invention to provide illuminants, especially based on LEDs, in a form which enables the use thereof in different fields of application. The properties of the materials should be such that they effectively withstand different environmental influences, whether they be physical, chemical or biological, over a long period. Moreover, the materials should be selected such that flexible moldings of any shape and dimensions can be obtained by means of simple processes. It was a further object to effectively protect not only the individual illuminants but also electrical connections between the illuminants and contacts to power sources. It was a further object to minimize scatter losses within the shaped body.

### SUMMARY OF THE INVENTION

The invention provides moldings which comprise a plurality of illuminants embedded in a silicone matrix, which silicone matrix is formed from an inner, soft silicone matrix A which is surrounded by one or more, harder silicone matrices B, and the silicone matrix B is optionally coated with a topcoat C.

### DETAILED DESCRIPTION OF THE INVENTION

The shape of the illuminant-silicone moldings is as desired and is matched to the particular application. They may be present in the form of pipes, as tubes, ribbons, as sheets or in the form of mats. Preference is given to pipes or ribbons equipped with a plurality of illuminants, especially a plurality of LEDs, these illuminants in a particularly preferred embodiment being arranged offset from one another (for example in a twisted manner), in such a way that they completely and homogeneously illuminate the surrounding space.

Suitable illuminants are any desired radiation-emitting components such as incandescent filament lamps, halogen lamps, low-pressure discharge lamps, high-pressure discharge lamps, fluorescent luminaires, neon luminaires, and injection luminescence lamps, preferably radiation-emitting semiconductor components of organic or inorganic semiconductors, known as LEDs. The LEDs may already be diodes encapsulated with plastic, usually silicone, or unencapsulated diodes.

Suitable LED illuminants are those which radiate in the infrared region, in the visible region or in the UV region. The selection depends on the intended use. The LED illuminants embedded in the matrix may emit at the same wavelength.

However, it is also possible to combine LED illuminants with different radiation characteristics with one another.

In general, a plurality of illuminants, especially LEDs, are conductively connected to one another, in series and/or in parallel. The illuminant arrangement may be connected to sensors, and to measurement/control devices. The number of illuminants and the arrangement thereof with respect to one another depends on the use thereof. The LED illuminants may be operated in a continuous or pulsed manner.

The arrangement of the illuminants, including the electrically conductive connections between the illuminants, and preferably also the contacts of the illuminant arrangement, are completely embedded in the inner silicone matrix A, and the latter by the outer silicone matrix B. The moldings are notable in that no further medium, for example air or water, is present between the illuminant and the silicone matrix A, or between silicone matrix A and silicone matrix B.

The silicone matrix in the form of the constituents, silicone matrix A and silicone matrix B, is highly transparent, the transmission in the range of a wavelength of 290-1200 nm>50%. Preference is given to a transmission of >85% in the wavelength range from 300 to 800 nm. Particular preference is given to a transmission of >90% in the wavelength range from 380 to 750 nm.

The silicone matrix A is optimized for the illuminant and should, in addition to the protective function for the electronic components (shock absorption), optimize the light yield (refractive index matching) and facilitate heat removal. Moreover, it is of significance that the illuminants remain permanently enclosed by the silicone matrix over the entire operating time of the illuminants, and air and water inclusions which lead to diffuse light scattering effects are prevented.

The inner silicone matrix A is soft with a Shore A hardness (DIN 53 505/ISO 868) of less than or equal to 10, or, if it is a liquid silicone oil, an average viscosity (at 23° C. and 1013 mbar) of 1 to 100×10⁶ mPa·s. The Shore A hardness is preferably less than 5, or the average viscosity (at 23° C. and 1013 mbar) is from 10 to 10×10⁶ mPa·s.

Suitable materials for the inner silicone matrix A are silicone oils, which are generally dialkylpolysiloxanes of the R₃SiO[—SiR₂O]ₙ—SiR₃ structure with a chain length of n>2. The alkyl radicals R may be the same or different and generally have 1 to 4 carbon atoms and may optionally be substituted. Some of the alkyl radicals R may also be replaced by other radicals, preferably by aryl radicals, which are optionally substituted, or by trialkylsiloxy groups in the case of branched silicone oils. Examples are methylsilicone oils (CH₃)₃SiO[—Si(CH₃)₂O]ₙ—Si(CH₃)₃, methylphenylsilicone oils (CH₃)₃SiO[—Si(CH₃)₂O]_{n′}—[—Si(C₆H₅)₂O]_{n″}—Si(CH₃)₃ or (CH₃)₃SiO[—Si(CH₃)₂O]_{n′}—[—Si(CH₃)(C₆H₅)O]_{n″}—Si(CH₃)₃ where, in each case, n′+n″>2, branched methylsilicone oils (CH₃)₃SiO[—Si(CH₃)(OSi(CH₃)₃)O]ₙ—Si(CH₃)₃, branched methylphenylsilicone oils (CH₃)₃SiO [—Si(C₆H₅)(OSi(CH0₃)O]ₙ—Si(CH₃)₃. By the introduction of aryl groups and the adjustment of the ratio of alkyl to aryl groups, the person skilled in the art can match the refractive index of the silicone matrix to the illuminant in a known manner. In addition, it is also possible with preference to use (“uncapped”) polydimethylsiloxane oils functionalized on the end groups. Such silicone oils are commercially available and preparable by known methods. Examples of commercially available silicone oils are the Wacker silicone oils from Wacker Chemie AG.

Silicone gels are also suitable for the inner silicone matrix A. Silicone gels are produced from two pourable components which crosslink at room temperature in the presence of a catalyst. One of the components generally consists of dialkylpolysiloxanes of the R₃SiO[—SiR₂O]ₙ—SiR₃ structure where n≧0, generally with 1 to 4 carbon atoms in the alkyl radical, where some or all of the alkyl radicals may be replaced by aryl radicals such as the phenyl radical, and one of the terminal R radicals at one or both ends is replaced by a polymerizable group such as the vinyl group. It is equally possible for some R radicals in the siloxane chain, also in combination with the R radicals of the end groups, to be replaced by polymerizable groups. Preference is given to using vinyl end-capped polydimethylsiloxanes of the CH₂═CH₂—R₂SiO[—SiR₂O]ₙ—SiR₂—CH₂═CH₂ structure.

The second component comprises an Si—H-functional crosslinker. The polyalkylhydrosiloxanes typically used are copolymers formed from dialkylpolysiloxanes and polyalkylhydrosiloxanes with the general formula R′₃SiO[—SiR₂O]ₙ—[SiHRO]ₘ—SiR′₃ where m≧0, n≧0, with the proviso that at least two SiH groups must be present, where R′ may be defined as H or R. There are accordingly crosslinkers with pendant and terminal SiH groups, while siloxanes where R′═H which possess only terminal SiH groups can also be used for chain extension. The crosslinking catalyst present is a small amount of an organoplatinum compound. The mixing of the components triggers the crosslinking reaction and forms the gel.

This crosslinking reaction can be accelerated by the action of heat and/or by means of electromagnetic radiation, preferably UV radiation. UV LEDs themselves can induce the crosslinking reaction of gels. The silicone gels are particularly soft materials, more particularly with a Shore 00 hardness of less than 50 (DIN 53 505/ISO 868), most preferably those with a penetration value to DIN ISO 2137 of >10 mm/10 (with 9.38 g quarter-cone and action time 5 s). Suitable silicone gels are commercially available, for example under the WACKER SilGel® trade name from Wacker Chemie AG, Munich.

For the inner silicone matrix A, the silicone oils and silicone gels mentioned are preferred.

The exact composition of the silicone matrix A depends on the radiation characteristics of the LEDs used, and the silicone materials used are those which result in optimal transparency in the appropriate wavelength range. What is important here is that there is permanent contact between the inner silicone matrix A and the illuminant, since any delamination phenomena are undesired owing to the associated significantly reduced light yields. For this reason, preferred materials for the inner silicone matrix A are self-adhesive systems or those with high surface tack. The materials for the silicone matrix A can also be optimized by means of addition of additives. Examples thereof are luminescent additives for light wavelength matching, metal or metal oxide particles or similar microparticles for varying the refractive index, for optimizing the light yield (for example using reflective particles which bring about homogeneous scatter of the light emitted by the illuminant outward) and for optimizing heat removal. To adjust the mechanical and rheological properties, it is optionally also possible to use small amounts of finely divided silica or fillers which strengthen the silicone elastomer matrix, for example silicone resin particles consisting of MT, MQ or DT units. The layer thickness of the inner silicone matrix A is matched to the particular application and is generally between 0.5 and 50 mm, preferably between 1 and 20 mm.

The outer silicone matrix B is optimized to the corresponding application, especially with regard to mechanical strength, and chemical, biological and physical (soil repellency, scratch resistance) resistance, while retaining the necessary transparency.

The outer silicone matrix B may, apart from the silicone oils and silicone gels mentioned, consist of the same silicone types as the inner silicone matrix A, with the difference that the Shore A hardness of the outer silicone matrix B is more than 10, and, in the case that the inner silicone matrix A likewise consists of a silicone type which is solid under standard conditions (23/50 DIN 50014), the difference between the Shore A hardnesses of inner silicone matrix A and outer silicone matrix B is at least 5, preferably at least 10, especially at least 20.

Silicones preferred for the silicone matrix B, and silicones optionally also usable for the silicone matrix A under the conditions mentioned (relatively low Shore A hardness), are crosslinked silicone rubbers which crosslink by condensation or addition reactions or by a free-radical mechanism. The crosslinking reaction can be initiated cationically, by means of appropriate catalysts, or free-radically, by means of peroxides, or by means of radiation, especially UV radiation, or thermally. Systems which lead to crosslinked silicone rubbers are commercially available preferably as one- or two-component systems, but also as multicomponent systems. Also suitable are silicone hybrid polymers.

Condensation-crosslinking silicone rubber systems contain

a) organopolysiloxanes having condensable end groups,

b) organosilicon compounds optionally having at least three hydrolyzable groups bonded to silicon per molecule, and

c) condensation catalysts.

Suitable crosslinking silicone rubbers which crosslink by condensation reaction are room temperature crosslinking one-component systems, known as RTV-1 silicone rubbers. The RTV-1 silicone rubbers are organopolysiloxanes with condensable end groups which crosslink in the presence of catalysts with condensation at room temperature. The most commonly used are dialkylpolysiloxanes of the R₃SiO[—SiR₂O]ₙ—SiR₃ structure with a chain length of n>2. The alkyl radicals R may be the same or different and generally have 1 to 4 carbon atoms and may optionally be substituted. Some of the alkyl radicals R may also be replaced by other radicals, preferably by aryl radicals, which are optionally substituted, in which case some of the alkyl (aryl) groups R are exchanged for groups capable of condensation crosslinking, for example alcohol, acetate, amine or oxime radicals. The crosslinking is catalyzed by means of suitable catalysts, for example tin or titanium catalysts. Suitable RTV-1 silicone rubbers are commercially available, for example the appropriate types of the ELASTOSIL® A, E or N series from Wacker Chemie AG.

Suitable crosslinked silicone rubbers which crosslink by condensation reaction are room temperature crosslinking two-component systems, known as RTV-2 silicone rubbers. RTV-2 silicone rubbers are obtained by means of condensation crosslinking of organopolysiloxanes polysubstituted by hydroxyl groups in the presence of silicic esters. The crosslinkers used may also be alkyl silanes with alkoxy, oxime, amine or acetate groups, which crosslink with the hydroxyl-terminated polydialkylsiloxanes in the presence of suitable condensation catalysts, for example tin or titanium catalysts. Suitable condensation-crosslinking RTV-2 silicone rubbers are commercially available, for example the appropriate types from the ELASTOSIL® RT series from Wacker Chemie AG.

Examples of the polydialkylsiloxanes present in RTV-1 and RTV-2 silicone rubber are those of the formula (OH)R₂SiO[—SiR₂O]ₙ—SiR₂(OH) with a chain length of n>2, where the alkyl radicals R may be the same or different, generally contain 1 to 4 carbon atoms and may optionally be substituted. Some of the alkyl radicals R may also be replaced by other radicals, preferably by aryl radicals, which are optionally substituted. The polydialkylsiloxanes preferably contain terminal OH groups which crosslink with the silicic esters or the alkylsilane/tin (titanium) catalyst system at room temperature.

Examples of the alkylsilanes which have hydrolyzable groups and are present in RTV-1 and RTV-2 silicone rubbers are those of the formula RₐSi(OX)₄₋ₐ where a=1 to 3 (preferably 1), and X is defined as R″ (alkoxy crosslinker), C(O)R″ (acetate crosslinker), N═CR″₂ (oxime crosslinker) or NR″₂ (amine crosslinker), where R″ is a monovalent hydrocarbon radical having 1 to 6 carbon atoms.

Addition-crosslinking silicone rubber systems contain

a) organosilicon compounds which have radicals with aliphatic carbon-carbon multiple bonds,

b) optionally organosilicon compounds with Si-bonded hydrogen atoms or, instead of a) and b),

c) organosilicon compounds which have radicals with aliphatic carbon-carbon multiple bonds and Si-bonded hydrogen atoms,

d) catalysts which promote the addition of Si-bonded hydrogen onto aliphatic multiple bonds and

e) optionally agents which retard the addition of Si-bonded hydrogen onto aliphatic multiple bonds at room temperature.

Suitable crosslinked silicone rubbers which crosslink by addition reaction are room temperature crosslinking two-component systems, known as addition-crosslinking RTV-2 silicone rubbers. Addition-crosslinking RTV-2 silicone rubbers are obtained by crosslinking, catalyzed by Pt catalysts, of organopolysiloxanes substituted by polyethylenically unsaturated groups, preferably vinyl groups, with organopolysiloxanes polysubstituted by Si—H groups, in the presence of platinum catalysts.

Preferably, one of the components consists of dialkylpolysiloxanes of the R₃SiO[—SiR₂O]ₙ—SiR₃ structure where n≧0, generally with 1 to 4 carbon atoms in the alkyl radical, where some or all of the alkyl radicals may be replaced by aryl radicals such as the phenyl radical, and one of the terminal R radicals at one or both ends is replaced by a polymerizable group such as the vinyl group. It is equally possible for some R radicals in the siloxane chain, also in combination with the R radicals of the end groups, to be replaced by polymerizable groups. Preference is given to using vinyl end-capped polydimethylsiloxanes of the CH₂═CH₂—R₂SiO[—SiR₂O]ₙ—SiR₂—CH₂═CH₂ structure.

The second component comprises an Si—H-functional crosslinker. The polyalkylhydrosiloxanes typically used are copolymers formed from dialkylpolysiloxanes and polyalkylhydrosiloxanes with the general formula R′₃SiO[—SiR₂O]ₙ—[SiHRO]ₘ—SiR′₃ where m≧0, n≧0, with the proviso that at least two SiH groups must be present, where R′ may be defined as H or R. There are accordingly crosslinkers with pendant and terminal SiH groups, while siloxanes where R′═H which possess only terminal SiH groups can also be used for chain extension.

The crosslinking catalyst present is a small amount of an organoplatinum compound.

Suitable RTV silicone rubbers are commercially available, for example the appropriate types from the ELASTOSIL® RT or ELASTOSIL® LR (LSR silicone rubber) or SEMICOSIL® series from Wacker Chemie AG.

Suitable silicone rubbers which crosslink by a free-radical mechanism or by addition reaction are solid silicone rubbers which crosslink on temperature increase (HTV).

Addition-crosslinking HTV silicone rubbers are obtained by the crosslinking of organopolysiloxanes substituted by polyethylenically unsaturated groups, preferably vinyl groups, with organopolysiloxanes polysubstituted by Si—H groups, in the presence of platinum catalysts.

Preferably, one of the components of the peroxidically crosslinking or addition-crosslinking HTV silicone rubbers consists of dialkylpolysiloxanes of the R₃SiO[—SiR₂O]ₙ—SiR₃ structure where n≧0, generally with 1 to 4 carbon atoms in the alkyl radical, where some or all of the alkyl radicals may be replaced by aryl radicals such as the phenyl radical, and one of the terminal R radicals at one or both ends is replaced by a polymerizable group such as the vinyl group. However, it is also possible to use polymers with pendant or pendant and terminal vinyl groups. Preference is given to using vinyl end-capped polydimethylsiloxanes of the CH₂═CH₂—R₂SiO[—SiR₂O]ₙ—SiR₂—CH₂═CH₂ structure, and vinyl end-capped polydimethylsiloxanes of the structure mentioned which also bear pendant vinyl groups. In the case of addition-crosslinking HTV silicone rubbers, the second component is a copolymer formed from dialkylpolysiloxanes and polyalkylhydrosiloxanes with the general formula R′₃SiO[—SiR₂O]ₙ—[SiHRO]ₘ—SiR′₃ where m≧0, n≧0, with the proviso that at least two SiH groups must be present, where R′ may be defined as H or R. There are accordingly crosslinkers with pendant and terminal SiH groups, while siloxanes where R′═H which possess only terminal SiH groups can also be used for chain extension. The crosslinking catalysts used are platinum catalysts.

HTV silicone rubbers are also processed as one-component systems, in which case the crosslinking reaction is induced by increasing the temperature and in the presence of peroxides as crosslinking catalysts, for example acyl, alkyl or aryl peroxides. Peroxide-crosslinking HTV silicone rubbers are obtained by the crosslinking of organopolysiloxanes optionally polysubstituted by ethylenically unsaturated groups, preferably vinyl groups. Suitable HTV silicone rubbers are commercially available, for example the appropriate ELASTOSIL® R or ELASTOSIL® R plus types from Wacker Chemie AG.

Additionally commercially available as of recently are specific HTV and RTV-1 silicone rubbers which are crosslinked via the addition reaction described, by virtue of specific platinum complexes or platinum/inhibitor systems thermally and/or photochemically activating and hence catalyzing the crosslinking reaction. Such systems are available, for example, as ELASTOSIL® R types, ELASTOSIL® RT types and Semicosil® types from Wacker Chemie AG.

Suitable materials are also silicone hybrid polymers. Silicone hybrid polymers are copolymers or graft copolymers of organopolymer blocks, for example polyurethane, polyurea or polyvinyl esters, and silicone blocks, generally based on polydialkylsiloxanes of the abovementioned specification. For example, thermoplastic silicone hybrid polymers are described in EP 1412416 B1 and EP 1489129 B1, the disclosure of which in this context shall also be included in the subject matter of this application. Such silicone hybrid polymers are referred to as thermoplastic silicone elastomers (TPSE) and are commercially available, for example the appropriate GENIOMER® types from Wacker Chemie AG.

Silicone resins are likewise suitable materials for the outer silicone matrix B. In general, the silicone resins contain units of the general formula R_{b}(RO)_{c}SiO_{(4-b-c)/2} in which

b is 0, 1, 2 or 3,

c is 0, 1, 2 or 3,

with the proviso that b+c≦3,

and R is as defined above,

which form a highly crosslinked organosilicone network. Suitable silicone resins are commercially available, for example the appropriate SILRES® types from Wacker Chemie AG.

Finally suitable for the silicone matrix B are also radiation-curing acryloyl-, epoxy- or vinyl ether-functional silicones, which are cured with free-radical formers or cationic photoinitiators.

Preferred materials for the outer silicone matrix B are the RTV-2 silicone rubbers mentioned, HTV silicone rubbers and the silicone hybrid polymers.

The properties of the silicone matrix B can be optimized by modification with additives, for example by the use of biocides such as algicides, bactericides or fungicides. By the addition of fillers, it is possible to adjust the hardness, though it should be ensured in the selection of the filler that the transparency is not restricted, which is why fillers based on silicone resin particles or nanoparticles are preferred.

The inner silicone matrix A may be surrounded by one or more outer silicone matrices B. The layer thickness of the outer silicone matrix B depends on how many silicone matrices B are formed, and on the use of the illuminant-silicone moldings, and is generally between 0.1 and 50 mm.

The illuminant-silicone molding may optionally also be provided with a topcoat C, which is applied to the silicone matrix B or to the outermost silicone matrix B. The topcoat serves to improve the scratch resistance and/or soil repellency and/or the antistatic properties. Preference is given to using the silicone resins mentioned as the topcoat C.

A more detailed overview of silicones, and the chemistry, formulation and performance properties thereof, can be found, for example, in Winnacker/Küchler, “Chemische Technik: Prozesse and Produkte, Band 5: Organische Zwischenverbindungen, Polymere”, pages 1095-1213, Wiley-VCH Weinheim (2005).

The inner silicone matrix A can be produced, for example, by means of filling, potting or transfer molding, according to the shape of the illuminant-silicone molding. The silicone matrix B can be produced, for example, by means of potting, extrusion, cast molding, compression molding or injection molding, according to the shape of the illuminant-silicone molding. The optional coating with a topcoat can be effected, for example, by means of coating, for example dipping, pouring, painting or spraying.

To produce a pipe-shaped molding, the procedure may be, for example, to fill a silicone rubber pipe (silicone matrix B) equipped with an LED chain with a silicone gel as silicone matrix A without bubbles under reduced pressure, then to crosslink the latter and subsequently to seal the LED-silicone matrix pipe thus obtained with an RTV silicone rubber. The procedure in the production of pipe-shaped illuminant-silicone moldings may also be to introduce a silicone oil as the silicone matrix A instead of a silicone gel. The remaining steps are performed analogously to the procedure with silicone gel. A further alternative for production of pipe-shaped moldings consists in processing silicone gel as the silicone matrix A and HTV silicone rubber as the silicone matrix B by the extrusion process.

To produce flat illuminant-silicone moldings, an HTV silicone rubber as the silicone matrix B is processed by means of compression molding or transfer molding to give a half-shell. In this half-shell are deposited commercial LED chains or commercial plastic bars equipped with LEDs in any desired arrangement. Subsequently, a silicone gel is poured in such that the LEDs are enclosed completely by silicone gel, which forms the silicone matrix A. The silicone gel is crosslinked and degassed, and the molding is concluded with a cover sheet made of the same material as the half-shell.

Another, alternative procedure is to produce a sheet of HTV silicone by means of compression or transfer molding, and then to provide this sheet at the edges with a firm RTV silicone rubber which is then crosslinked to form a solid land, in order to obtain the half-shell geometry. The remaining steps correspond to the above procedure.

A further alternative for production of flat parts consists in working by the injection molding process and using LSR silicone rubber for the production of the half-shell or sheets.

In the case of use of silicone hybrid polymers, an extruder can be charged with an illuminant chain, and the silicone hybrid polymer can be extruded to form a molding which envelops the chain, the intermediate space between chain and silicone hybrid molding being simultaneously filled with silicone oil.

The inventive illuminant-silicone moldings are notable for their variety of uses. In general, it is thus possible to introduce any light unit in protected form into different applications.

In the production of LED-silicone moldings, a significant advantage is that it is possible to dispense with the encapsulation of LEDs with silicone beforehand, and to process unencapsulated LEDs as illuminants.

The three main fields of use are mere illumination for the purposes of decoration and of safety, the initiation of chemical or biological reactions, and marking under difficult conditions.

Conceivable applications for the purpose of decoration would be illuminated ice, LED-silicone moldings woven into carpet, in aquaria, saunas or pool/bath illuminations, as illuminated decorative joints and in any form of illuminated adornments. Applications for safeguarding life and health are possible in lifejackets, on the edges of steps, in and on sports equipment and in motor vehicles.

Chemical reactions and/or biological reactions can be triggered, for example, by specific wavelengths of light in the ultraviolet, visual or infrared range. When the suitable LED illuminant is incorporated into the silicone matrices, it is possible to trigger growth reactions of plants or fruits, for example in greenhouses or dark spaces, the killing of plants and microorganisms, for example by means of dermatological UV vests or covers, or the simple heating of reactive mixtures. The high inertness of the silicone body allows this to be performed in acidic, basic, salty, mechanically stressed and sterilized media.

Marking in the manner of a guiding principle can be implemented in ship and harbor lighting, also under water, in mobile airstrip boundaries, in the designation of pipes, tubes or cables in refineries, as angling equipment and on signs.

The inventive illuminant-silicone moldings are preferably employed under moist conditions or directly in liquids. Particular preference is given to illuminant-silicone moldings which are equipped with UV LEDs and are used in aqueous media to irradiate vegetable materials or to disinfect water.

When illuminant-silicone moldings, especially LED-silicone moldings, of specific wavelength are introduced into bioreactors for production of biomasses, especially for growing algae, it is possible to considerably enhance the growth of the algae through the penetration depth of the light and through pulsing of the light to obtain light and dark zones. In addition, UV LED-silicone moldings can be used to disinfect water-filled containers, for example in medical equipment, emulsifying systems, tubes and pipes. In combination with a solar cell, the provision of a mobile drinking water processing plant is possible.

The illuminant-silicone moldings provide radiative bodies which are usable universally owing to the resistance of silicones to a wide variety of different external influences. A great advantage of the modular construction based on an inner silicone matrix A and at least one outer silicone matrix B is that it becomes possible to optimize the molding with regard to light yield and resistance. While the inner layer is optimized for light yield, heat removal and shock absorption, the outer silicone matrix B is optimized for mechanical strength, and chemical and biological, physical (soil repellency, scratch resistance) resistance with retention of the necessary transparency. This would not be achievable in this quality with a one-layer structure owing to the interactions which occur between the additives required, since the profile of requirements for the silicone component close to the illuminant is quite different to the profile of requirements on the silicone component with contact to the surrounding medium.

### Example 1

A silicone pipe of length approx. 1 m, produced by extrusion from a high-transparency addition-crosslinking solid silicone rubber (ELASTOSIL® R plus 4305/50, Wacker Chemie AG) with a Shore A hardness of 50, an external diameter of 12 mm and a wall thickness of 2 mm was hung up vertically in a vacuum chamber, and the lower end of the pipe was sealed tight with a silicone stopper. Subsequently, an LED module bar of length 1 m with 60 LEDs (from LED1.de, Dessau), which emit, at a voltage of 12 V, light of the preferred wavelength of 400 nm at an emission angle of 120° with a brightness of in each case 100 mcd, was introduced cautiously into the silicone pipe such that the connection cable and the plug with which the LED module bar had been provided beforehand projected out of the upper end of the pipe. This arrangement was then potted under reduced pressure with a room temperature vulcanizing addition-crosslinking silicone elastomer mixture (WACKER SilGel® 612 A/B) which has a mix viscosity of approx. 1000 mPa*s (at 23° C. and 1013 mbar), and crosslinked at room temperature within 24 hours to give a silicone gel with a penetration value (DIN ISO 2137) of about 300 mm/10. For this purpose, the two components of the silicone potting material were first mixed cautiously in a ratio of 1:1. The mixture thus obtained was subsequently evacuated together with the silicone pipe/LED bar arrangement in the vacuum chamber at a vacuum of below 50 mbar for 30 minutes in order to remove absorbed air, which can later lead to troublesome bubble formation in the vulcanizate. The degassed silicone potting material was introduced cautiously and under reduced pressure into the silicone pipe containing the LED bar and vulcanized at room temperature under reduced pressure for 24 hours. Thereafter, the silicone pipe which contains the LED bar now encapsulated in silicone gel was removed from the vacuum chamber. Finally, the silicone stopper mentioned at the outset was removed and the two ends of the silicone pipe were sealed with a firm, self-adhesive silicone rubber (Semicosil® 989/1K from Wacker Chemie AG), which crosslinks at 130° C. to give a transparent to opaque vulcanizate with a Shore A hardness of 55.

### Example 2

The procedure was analogous to example 1, but with the following differences:

The outer matrix material B used was a silicone pipe with a Shore A hardness of 40 (ELASTOSIL® R plus 4305/40, Wacker Chemie AG).

The inner matrix material A used was a silicone oil (WACKER® AK 100 000 silicone oil), consisting of dimethylsiloxy and trimethylsiloxy units with a viscosity of 100 000 mPa·s (at 23° C. and 1013 mbar).

The silicone oil was introduced through a cannula below the surface from the bottom upward, in order to prevent the introduction of air bubbles. Subsequently, any air inclusions were removed by evacuating the silicone oil-filled pipe at below 50 mbar for 10 minutes.

The silicone pipe ends were sealed, instead of with a one-component heat-curing silicone rubber, with a firm self-adhesive RTV-1 silicone rubber (ELASTOSIL® E47 from Wacker Chemie AG), which crosslinks at room temperature through atmospheric humidity to give a transparent to opaque vulcanizate with a Shore A hardness of 35.

### Example 3

The procedure was analogous to example 1, except that, instead of the silicone pipe consisting of solid silicone rubber, a pipe which was produced by extrusion from thermoplastic silicone elastomer and had the Shore A hardness of 50 was used. The high-transparency thermoplastic base material which contains urea groups and is used for that purpose possesses a siloxane content of 92% by weight, a weight-average molecular weight MW of approx. 120 000 g/mol, a softening point of approx. 125° C., a melt viscosity at 170° C. of about 100 000 Pa·s, and is obtainable from Wacker Chemie AG under the trade name GENIOMER® 140. In addition, the pipe ends, after potting with the silicone gel, were not sealed with a one-component heat-curing silicone, but with a firm self-adhesive RTV-1 silicone rubber which crosslinks at room temperature through atmospheric humidity to give a transparent to opaque vulcanizate with a Shore A hardness of 35 (ELASTOSIL® E 47, Wacker Chemie AG).

### Example 4

The procedure was analogous to example 3, except that, instead of the silicone gel used there as the internal silicone matrix A, a silicone oil (WACKER® AK 1000 silicone oil) consisting of dimethylsiloxy and trimethylsiloxy units with a viscosity of 1000 mPa·s (at 23° C. and 1013 mbar) was used.

### Example 5

The procedure was analogous to example 1, except that, instead of the potting material described in example 1, a UV-activatable silicone potting material (Semicosil® 912 UV from Wacker Chemie AG), which crosslinks to give a soft silicone gel, was used for the inner silicone matrix A. The two-component material had, after the mixing of the two components in a ratio of 9:1, a mixed viscosity of 1000 mPa·s (at 23° C. and 1013 mbar), and afforded a vulcanizate with gel-like softness and a penetration value (DIN ISO 2137) of about 70 mm/10 (9.38 g quarter-cone). In contrast to example 1, the UV-activatable silicone potting material was vulcanized by exposure in a Höhnle UVA cube (radiation intensity: 140 mW/cm²) for 5 seconds, and subsequently leaving it to stand at room temperature without UV irradiation. After 5 minutes, the material was crosslinked through to form a silicone gel.

### Example 6

A transparent half-shell (external dimensions: 1000×500×20 mm; wall thickness: 10 mm; depth of the recess: 10 mm), consisting of a silicone elastomer with a Shore A hardness of 50 and produced by the cast molding process (a pourable, room temperature vulcanizable two-component mixture (ELASTOSIL® LR 7661 A/B, Wacker Chemie AG) which, after mixing of the two components in a ratio of 9:1, had a mixed viscosity of about 18 000 mPa·s and crosslinked within 5 minutes at 165° C. to give a clear transparent vulcanizate was used) was placed flat into a vacuum chamber with the recess upward. Subsequently, four LED modules of the type described in example 1, each of length 50 cm and connected in parallel to one another, were placed alongside one another at equal distances into the recess of the half-shell such that connection cables along with plugs with which the LED modules have been provided come to rest beside the half-shell. This arrangement was potted with the two-component silicone (WACKER SilGel® 612 A/B) which was described in example 1 and crosslinks to form a gel, and in the same manner as described above. In doing this, it should be ensured that the half-shells are completely filled with the potting material and the LED bars are completely enclosed by the potting material. On completion of vulcanization of the encapsulation material under reduced pressure and at room temperature, the half-shell was removed from the vacuum chamber. Subsequently, the upper edge of the half-shell was painted with a firm self-adhesive RTV-1 silicone rubber (ELASTOSIL® E 47, Wacker Chemie AG) which crosslinks at room temperature through atmospheric humidity to give a transparent to opaque vulcanizate with the Shore A hardness of 35. Thereafter, a transparent sheet with dimensions 1000×500×10 mm and composed of the same silicone elastomer as the half-shell was placed on flush. Embedded air was removed by smoothing out the air bubbles at the side, and vulcanization was effected at room temperature for 24 hours.

1. An illuminant-silicone molding which comprises a plurality of illuminants embedded in a silicone matrix, which silicone matrix is formed from an inner, soft silicone matrix A which is surrounded by one or more, harder silicone matrices B.

2. The illuminant-silicone molding as claimed in claim 1, wherein the illuminants are LED illuminants.

3. The illuminant-silicone molding as claimed in claim 1, wherein a plurality of illuminants, conductively connected to one another, are connected in series and/or in parallel.

4. The illuminant-silicone molding as claimed in claim 1, wherein the illuminants emit at the same wavelength.

5. The illuminant-silicone molding as claimed in claim, wherein illuminants with different radiation characteristics are combined with one another.

6. The illuminant-silicone molding as claimed in claim 1, wherein the inner silicone matrix A has a Shore A hardness less than or equal to 10, or a viscosity of 1 to 100×10⁶ mPa·s at 23° C. and 1013 mbar.

7. The illuminant-silicone molding as claimed in claim 1, wherein the Shore A hardness of the outer silicone matrix B is more than 10, and, in the case that the inner silicone matrix A likewise consists of a silicone type which is solid under standard conditions, the difference between the Shore A hardnesses of the inner silicone matrix A and the outer silicone matrix B is at least 5.

8. The illuminant-silicone molding as claimed in claim 1, wherein silicone oils or silicone gels are used for the inner silicone matrix A.

9. The illuminant-silicone molding as claimed in claim 1, wherein RTV-2 silicone rubbers or HTV silicone rubbers or silicone hybrid polymers are used for the outer silicone matrix B.

10. The illuminant-silicone molding as claimed in claim 1, wherein the illuminant-silicone molding is also equipped with a topcoat.

11. The illuminant-silicone molding as claimed in claim 1, wherein the molding is in the form of pipes, tubes, ribbons, sheets or in the form of mats.

12. The illuminant-silicone molding as claimed in claim 1, wherein the illuminants within the silicone matrix A are arranged offset from one another in such a way that they completely and homogeneously illuminate the surrounding space.

13. A process for producing illuminant-silicone moldings as claimed in claim 1, wherein the illuminant-silicone moldings are equipped with an inner, soft silicone matrix A, and surrounded by one or more, harder silicone matrices B.

14. A method of providing illumination comprising operating an illuminant-silicone as claimed in claim 1.

15. A method of initiating a chemical or biological reaction, comprising providing to a reaction mixture light from an illuminant-silicone molding as claimed in claim 1.

16. A method of disinfecting water, comprising exposing the water to light from an illuminant-silicone molding as claimed in claim.

17. A method of operating a bioreactor to produce a biomass, comprising providing light from an illuminant-silicone molding as claimed in claim 1 to the bioreactor.

18. A mobile drinking water processing plant comprising an illuminant-silicone molding as claimed in claim 1 in combination with a solar cell.

19. The method of claim 17, wherein the biomass comprises algae.
